(19) United States
(12) Patent Application Publication
(10) Pub. No.: US 2023/0192591 A1
(43) Pub. Date: Jun. 22, 2023

(54) PROCESS FOR PREPARING TRIENTINE DIHYDROCHLORIDE

(71) Applicants: Daniel J. COUGHLIN, West Deptford, NJ (US); Macfarlan Smith Limited, Edinburgh (GB)

(72) Inventors: Daniel J. COUGHLIN, West Deptford, NJ (US); Jeremy C WILT, Northborough, MA (US)

(21) Appl. No.: 17/907,512

(22) PCT Filed: Mar. 26, 2021
(86) PCT No.: PCT/US2021/070320

§ 371 Date: Sep. 27, 2022

Related U.S. Application Data
(60) Provisional application No. 63/000,931, filed on Mar. 27, 2020.

(51) Int. Cl. C07C 209/68

(52) U.S. Cl. CPC C07C 209/68

(57) ABSTRACT

The present invention relates to a process for preparing trientine dihydrochloride as an active pharmaceutical ingredient (API). The present invention relates to a process for the preparation of trientine dihydrochloride with a pharmaceutical grade of purity. The invention also relates to novel intermediates used in the preparation of trientine dihydrochloride.

### FIELD OF THE DISCLOSURE

The present invention relates to a process for preparing trientine dihydrochloride. The invention also concerns intermediates useful in the preparation of trientine dihydrochloride.

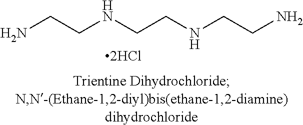

### BACKGROUND OF THE DISCLOSURE

Trientine hydrochloride is marketed in the United States under the trade name Syprine® that is indicated for the treatment of patients with Wilson's disease who are intolerant of penicillamine. Wilson's disease (hepatolenticular degeneration) is an autosomal inherited metabolic defect resulting in an inability to maintain a near-zero balance of copper.

### SUMMARY OF THE DISCLOSURE

The present specification describes a process for the preparation of trientine dihydrochloride with a pharmaceutical grade of purity. The process avoids the use of chromatographic purification. In certain embodiments, the process also avoids undesired reactions, such as polyalkylation, and further avoids the formation of acidic by-products. The process utilizes a protecting group strategy to more precisely control the equivalents of hydrochloride by relying on the difference in pKa of amides and tertiary amines to exclusively form a dihydrochloride salt without also forming trientine tetrahydrochloride salt or the free base. The process also provides for a single step for deprotection of all protecting groups. Further, the process utilizes chromophores on the process intermediates to allow for monitoring of the reaction progress by using HPLC.

### DETAILED DESCRIPTION OF THE DISCLOSURE

The following description is presented to enable a person of ordinary skill in the art to make and use the various embodiments. Descriptions of specific devices, techniques, and applications are provided only as examples. Various modifications to the examples described herein will be clear to those of ordinary skill in the art, and the general principles described herein may be applied to other examples and applications without departing from the spirit and scope of the various embodiments. Therefore, the various embodiments are not intended to be limited to the examples described herein and shown but are to be accorded the scope consistent with the claims.

### Terms/Definitions

### Abbreviations

atm atmosphere
CbzOSu 1-{[(benzyloxy)carbonyl]oxy}-2,5-pyrrolidinedione (N-(Benzyloxycarbonyloxy)succinimide))
EtOH ethanol
eq. equivalent
min minutes(s)
hr hour(s)
TBME tert-butyl methyl ether
vol volumes

As used herein and unless otherwise specified, the following terms in quotes have the definitions as noted below.

The term “acid addition salt” includes an inorganic or organic salt selected from the group consisting of a hydrobromide, hydrochloride, sulphate, bisulphate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, malonate, salicylate, propionate, methanesulphonate, ethanesulphonate, benzenesulphonate, or p-toluenesulphonate salt and the like.

The term “aromatic solvent” is selected from the group consisting of benzene and toluene, or a mixture thereof. Toluene is a particular aromatic solvent.

The term “inorganic base” means an alkali or alkaline earth base such as potassium, sodium, calcium or magnesium hydroxide or carbonate; particularly the inorganic base is sodium carbonate or potassium carbonate; more particularly the inorganic base is sodium carbonate.

The term “C₁₋₅ alcohol solvent” is selected from methanol, ethanol, propanol, butanol and pentanol, including isomers such as n-, i-, t-isomers, or a mixture thereof. Ethanol is a particular alcohol solvent.

The term “C₄H₁₀—O₅H₁₂ ether” is selected from ethyl ether, methyl propyl ether, methyl isopropyl ether, ethyl propyl ether, ethyl isopropyl ether, and methyl t-butyl ether (TBME), or a mixture thereof.

As used herein and unless otherwise specified, the terms “about” and “approximately,” when used in connection with a numeric value or a range of values which is provided to characterize a reaction, reactant, or conditions, e.g., a specific temperature or temperature range, including, e.g., melting, or evaporation, may deviate to an extent deemed reasonable to one of ordinary skill in the art. In certain embodiments, the term “about” or “approximately” means within 1, 2, 3 or 4 standard deviations. In certain embodiments, the term “about” or “approximately” means within 30%, 25%, 20%, 15%, 10%, 9%, 8%, 7%, 6%, 5%, 4%, 3%, 2%, 1%, or 0.5% of a given value or range.

The term “ambient temperature” means one or more room temperatures between about 15° C. to about 30° C., such as about 15° C. to about 25° C.

The term “overnight” refers to the period of time between the end of one working day to the subsequent working day in which a time frame of about 12 to about 18 hours has elapsed between the end of one procedural step and the instigation of the following step in a procedure.

The term “consisting” is closed and excludes additional, unrecited elements or method steps in the claimed invention.

The term “consisting essentially of” is semi-closed and occupies a middle ground between “consisting” and “comprising.” “Consisting essentially of” does not exclude additional, unrecited elements or method steps which do not materially affect the essential characteristic(s) of the claimed invention.

The term “comprising” is inclusive or open-ended and does not exclude additional, unrecited elements or method steps in the claimed invention. The term is synonymous with “including but not limited to.” The term “comprising” encompasses three alternatives, namely (i) “comprising” (ii) “consisting” and (iii) “consisting essentially of.”

The term “hydrogenation catalyst” includes a catalyst that effects amine deprotection both by N-debenzylation and cleavage of carbamate protecting moiety with hydrogen to yield the corresponding amine. A hydrogenation catalyst includes both heterogeneous and homogeneous catalysts.

The term “heterogeneous catalyst” is a heterogeneous platinum group metal (PGM) catalyst, for example, a heterogeneous palladium or platinum catalyst. Examples of palladium catalysts include but are not limited to colloidal palladium, palladium sponge, palladium plate or palladium wire. Examples of platinum catalysts include but are not limited to colloidal platinum, platinum sponge, platinum plate or platinum wire. The heterogeneous PGM metal catalyst may be a PGM on a solid support. The support may be selected from the group consisting of carbon, alumina, calcium carbonate, barium carbonate, barium sulfate, titania, silica, zirconia, ceria and a combination thereof. When the support is alumina, the alumina may be in the form of alpha-Al₂O₃, beta-Al₂O₃, gamma-Al₂O₃, delta-Al₂O₃, theta-Al₂O₃ or a combination thereof. When the support is carbon, the carbon may be in the form of activated carbon (e.g. neutral, basic or acidic activated carbon), carbon black or graphite (e.g. natural or synthetic graphite). An example of a heterogeneous PGM catalyst is palladium on carbon. The heterogeneous catalyst may be a water-wet catalyst. The quantity of water present may be about 50%. An example of another heterogeneous PGM catalyst is platinum on carbon. The catalyst loading may be up to about 10 mole %. In one embodiment, the catalyst loading may be in the range of about 0.1-1.0 mole %. While it is typically sufficient for a single charge of hydrogenation catalyst to be used in a reaction mixture, a second or further charge may be added and the hydrogenation continued if it has been determined (e.g. via in-process analysis) that the reaction has not gone to completion and starting material remains.

A hydrogenation as carried out herein is not limited relative to the pressure at which the hydrogenation is carried out. In this regard, the hydrogenation may be carried out with an initial hydrogen pressure in the range of up to about 5 atm. In a particular embodiment, the hydrogen pressure is about 2.5-3 atm.

Various further aspects and embodiments of the present invention will be apparent to those skilled in the art in view of the present disclosure. The use of “and/or” herein is to be taken as specific disclosure of each of the two specified features or components with or without the other. For example, “A and/or B” is to be taken as specific disclosure of each of (i) A, (ii) B, and (iii) A and B, just as if each is set out individually herein.

### EMBODIMENTS

Step A—Preparation of Benzyl (2-chloroethyl)carbamate

An embodiment of the invention includes the process of preparing benzyl (2-chloroethyl)carbamate by reacting 2-chloroethylamine hydrochloride with 1-{[(benzyloxy)carbonyl]oxy}-2,5-pyrrolidinedione (Cbz-OSu) to form benzyl (2-chloroethyl)carbamate as shown in the below scheme.

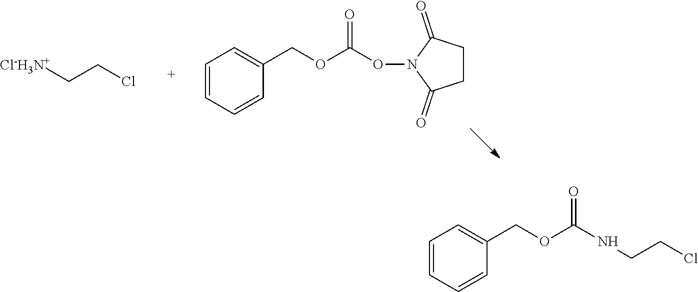

In one embodiment, the reaction is conducted by mixing Cbz-OSu in an aromatic solvent with an aqueous solution of 2-chloroethylamine hydrochloride, and then adding to the aforesaid mixture an aqueous inorganic base solution. The aromatic solvent may be selected from the group consisting of benzene and toluene, or a mixture thereof. In one embodiment, the aromatic solvent is toluene. In one embodiment, the inorganic base is selected from an alkali or alkaline earth base such as potassium, sodium, calcium or magnesium hydroxide or carbonate; particularly the base is sodium carbonate or potassium carbonate; more particularly sodium carbonate. In one embodiment, the molar equivalent (eq) ratio of the Cbz-OSu:2-chloroethylamine hydrochloride:inorganic base is about 1:1.1-1.3:1.1-1.4; provided that the eq amount of 2-chloroethylamine hydrochloride is equal to or less than the eq amount of inorganic base; and 1 wt (g) of Cbz-OSu:1.5-1.9; particularly about 1.7-1.8; more particularly about 1.75 volume (mL) of aromatic solvent. In another embodiment, the volume ratio of the aromatic solvent to total water in the reaction is about 1:2-3; particularly about 1:2.5. In an embodiment, the 2-chloroethylamine hydrochloride in water exists in about 60-80 wt %, particularly about 70 wt %. In an embodiment, the inorganic base in water exists in about 8-20 wt %, particularly about 12 wt %. In yet another embodiment, the volume ratio of the aromatic solvent comprising Cbz-OSu:aqueous solution of 2-chloroethylamine hydrochloride:aqueous inorganic base solution is about 7-4:1:10-14; particularly 5.5:1:12. In a particular embodiment, the addition of the aqueous inorganic base solution is over a period of time; particularly the inorganic base solution is added over the course of about 10-60 min, more particularly added dropwise. In one embodiment, the reaction is performed to keep the temperature about ≤−5-30° C.; particularly about ≤25° C., more particularly 15-25° C. In one embodiment, after completion of the addition of the base, the mixture is stirred at about 10-30° C.; particularly 15-25° C., for about 1-24 hrs; particularly for 2 hrs. After the stirring, the reaction mixture separates into an aqueous layer and an aromatic solvent layer. The aqueous layer is discarded from the aromatic solvent layer containing benzyl (2-chloroethyl)carbamate which is retained.

Step B—Preparation of Dibenzyl((Ethane-1,2-Diylbis(Benzylazanediyl))Bis(Ethane-2,1-Diyl))Dicarbamate Dihydrochloride

The invention includes a process comprising preparing dibenzyl ((ethane-1,2-diylbis(benzylazanediyl))bis-(ethane-2,1-diyl))dicarbamate dihydrochloride by reacting benzyl (2-chloroethyl)carbamate with N,N′-di-benzylethylenediamine, followed by treatment with HCl as shown in the following Scheme:

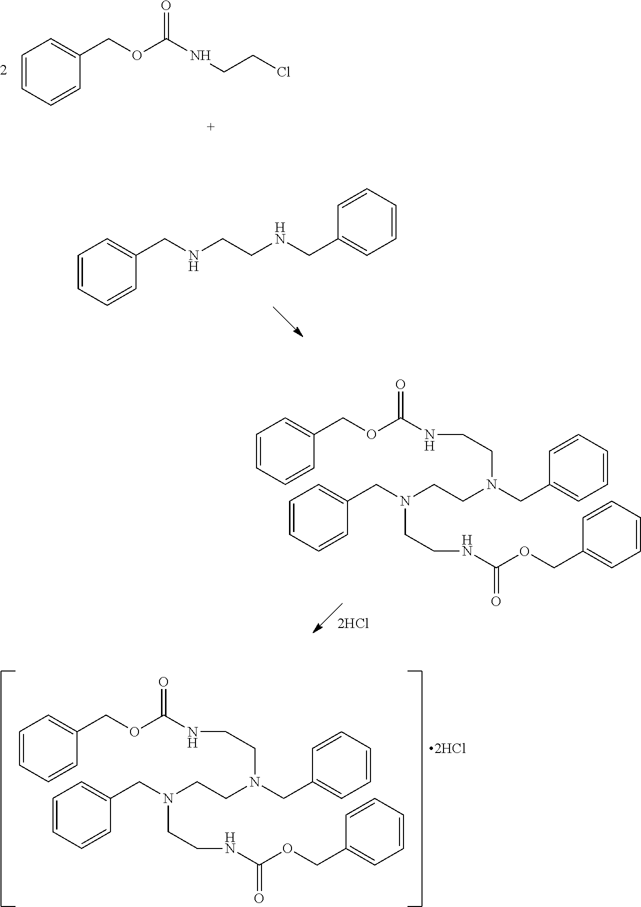

In one embodiment, the method for preparing dibenzyl ((ethane-1,2-diylbis(benzylazanediyl))bis(ethane-2,1-diyl))dicarbamate dihydrochloride involves preparing the N,N′-dibenzylethylenediamine from an acid addition salt of N,N′-dibenzylethylenediamine in situ. In a particular embodiment, the acid addition salt of N,N′-di-benzylethylenediamine is N,N′-dibenzylethylenediamine diacetate. In the situation where N,N′-dibenzyl-ethylenediamine is prepared in situ from an acid addition salt of N,N′-dibenzylethylenediamine, an inorganic base is utilized to neutralize the acid addition salt of N,N′-dibenzylethylenediamine to form the N,N′-dibenzylethylenediamine. In one embodiment, the reaction occurs by combining the acid addition salt of N,N′-dibenzylethylenediamine, benzyl (2-chloroethyl)carbamate in an aromatic solvent, and water, and then adding an aqueous inorganic base (about 10-20 wt %, particularly 15.3 wt %) thereto. In one embodiment, the inorganic base is selected from an alkali and alkaline earth base such as potassium, sodium, calcium or magnesium hydroxide or carbonate, particularly the inorganic base is sodium carbonate. In another embodiment, the molar eq ratio of the acid addition salt of N,N′-dibenzylethylenediamine:benzyl (2-chloroethyl)carbamate:inorganic base is about 1:2-2.6:2.2-2.6; particularly about 1:2-2.5:2.2-2.4, more particularly about 1:2.5:2.3. In particular, the inorganic base is used in an amount sufficient for the generation of N,N′-dibenzylethylenediamine free base by neutralizing the corresponding diacetic acid salt. When an alkali or alkaline earth carbonate base is employed, the reaction is evidenced by generation of CO₂. The aromatic solvent to total water volume ratio is about 1:1-2; particularly about 1:1.5.

In another embodiment, the reaction mixture is heated to about 80-100° C., particularly to about 90-100° C. to distill off the aromatic solvent, and the aromatic solvent is considered distilled off when the reaction mixture temperature reaches about 100° C. Following the removal of the aromatic solvent, the residual solution (aqueous) is stirred for about 15-30 hr, particularly about 20 hr, at about 100° C. The residual solution is then cooled to about 20-40° C., particularly to about 30° C., followed by the addition of aromatic solvent (about 2-4, particularly about 2.5, times the amount used in Step A) and then is cooled further to about 15-25° C., particularly to about 20° C., resulting in a biphasic aromatic/aqueous solvent mixture. The aromatic and aqueous solvent phases are separated, and the aqueous solvent phase is discarded. A C₁₋₅ alcohol, more particularly ethanol (about 2-4, particularly about 2.5, times the amount of aromatic solvent used in Step A) is added to the aromatic solvent phase (about 1:0.75-1.25, particularly 1:1 volume ratio of aromatic:alcohol solvents). The aromatic/alcoholic solvent mixture is then cooled to about −5-10° C., particularly to about 0-5° C., followed by the addition of aqueous HCl while maintaining the temperature at about ≤10° C. In one embodiment, the eq ratio of HCl:N,N′-dibenzylethylenediamine diacetate is 2:1. In another embodiment, the wt % of the HCl in water is about 35-40%; particularly about 37%. The aromatic/alcoholic solvent mixture is then stirred at about −5-10° C., particularly at about 0-5° C. for about an additional 1-4 hr, particularly about 2 hr, to precipitate dibenzyl ((ethane-1,2-diylbis(benzylazanediyl))bis(ethane-2,1-diyl))dicarbamate dihydrochloride. The precipitate is isolated, and optionally rinsed 2-3 times with a mixture of C₁₋₅ alcohol and aromatic solvent, particularly ethanol and toluene; more particularly 2 volumes (mL) of C₁₋₅ alcohol and aromatic solvent mixture:1 wt (g) of N,N′-di-benzylethylenediamine diacetate. In one embodiment, the wt % ratio of the alcohol to aromatic solvent is about 40%-55%, particularly about 45-51%, and more particularly about 48%. The isolated precipitate is dried optionally under vacuum and/or with heating, particularly at about ≥30° C., more particularly at about 50-60° C., further particularly at about 55° C.

Step C—Preparation of N,N′-(Ethane-1,2-Diyl)Bis(Ethane-1,2-Diamine) Dihydrochloride (Trietine Dihydrochloride)

Another embodiment, of the invention includes a process that comprises the preparation of trientine dihydrochloride from dibenzyl ((ethane-1,2-diylbis(benzylazanediyl))bis(ethane-2,1-diyl))dicarbamate dihydrochloride by hydrogenation deprotection as shown in the following Scheme:

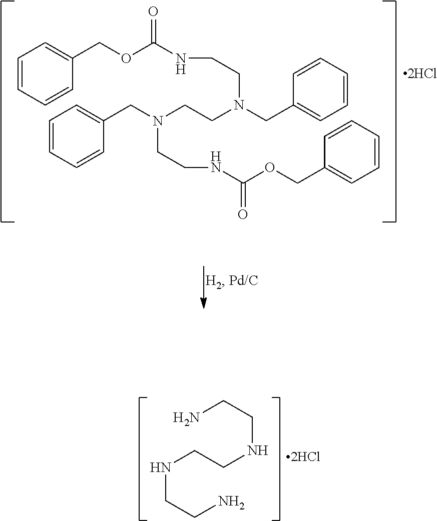

In one aspect, the invention provides for hydrogenation deprotection of a suspension of dibenzyl ((ethane-1,2-diylbis(benzylazanediyl))bis(ethane-2,1-diyl))dicarbamate dihydrochloride in a water and C₁₋₅ alcohol mixture (1 wt (g) of dibenzyl ((ethane-1,2-diylbis(benzylazanediyl))bis(ethane-2,1-diyl))dicarbamate dihydrochloride:6.67 volume (mL) of C₁₋₅ alcohol and water mixture. In one embodiment, the alcohol is ethanol. In one embodiment, the C₁₋₅ alcohol and water mixture has the wt % of 20-40% C₁₋₅ alcohol to water, particularly about 30%. In another embodiment, the hydrogenation deprotection uses a heterogeneous or homogeneous catalyst. In one embodiment, the hydrogenation catalyst is a heterogeneous catalyst. In a further embodiment, the hydrogenation catalyst is Pd/C. In another embodiment, the Pd/C is present as 10% w/50% H₂O (0.4 wt % anhydrous basis, 0.25 mol % Pd). In one embodiment, the reaction mixture is hydrogenated in a pressure vessel (e.g., Hastelloy Pressure Vessel). In another embodiment, the hydrogenation occurs at about 15-30° C., particularly at about 20-25° C. In another embodiment, the hydrogenation occurs for about 4-8 hr, particularly for about 6 hr. In another embodiment, the pressure applied during hydrogenation is up to 5 atm; particularly 2.5-3 atm. An optional embodiment is wherein the hydrogenation reaction is sampled and analysed for reaction completion by a method appreciated by one skilled in the art, for example using FIO ICP analysis (ICP-OES).

Following the hydrogenation, the solids, including the catalyst, are separated from the reaction mixture solution, and optionally, the separated solids are rinsed with a C₁₋₅ alcohol and water mixture at least once, and particularly twice, and the rinse(s) are combined with reaction mixture solution. In one embodiment, the C₁₋₅ alcohol and water mixture for rinse is 1 vol C₁₋₅ alcohol and water mixture per wt (g) of dibenzyl ((ethane-1,2-diylbis(benzylazanediyl))bis(ethane-2,1-diyl))dicarbamate dihydrochloride. In one embodiment, the water and C₁₋₅ alcohol mixture for rinse has the wt % of 20-40% C₁₋₅ water to alcohol, particularly about 30%. In one embodiment, the alcohol is ethanol. In another embodiment, the solids are separated by filtering. Another embodiment is wherein the filtering is carried out through about a≤0.2 μm filter. In another embodiment, the reaction mixture solution separated from the solids is treated with a metal scavenger, e.g., such as QuadraPure®, Quadrasil® or Isolute®. In one embodiment, the wt % of the metal scavenger is about 5-10 wt %, particularly about 6.5 wt %, relative to the theoretical yield in grams of the trientine dihydrochloride product. In another embodiment, the treatment with the metal scavenger is for about 15-20 hr, particularly 18 hr. In another embodiment, following the treatment with the metal scavenger, the reaction mixture is separated from any solids therein. In one embodiment, the separation of the scavenger is affected by filtering, particularly using a filter of about ≤0.45 μm. The isolated (filtered) reaction mixture solution (filtrate) is then concentrated to about 3 vol (1 wt (g) of dibenzyl ((ethane-1,2-diylbis(benzylazanediyl))bis(ethane-2,1-diyl))dicarbamate dihydrochloride:1 volume (mL) filtrate), then diluted with about 6 vol of C₁₋₅ alcohol (1 wt (g) of dibenzyl ((ethane-1,2-diylbis(benzylazane-diyl))bis(ethane-2,1-diyl))dicarbamate dihydrochloride:1 volume (mL) C₁₋₅ alcohol), and then concentrated again. In one embodiment, the C₁₋₅ alcohol is ethanol. The process of concentration and dilution is repeated one to three more times and then after the last concentration, a precipitate is formed. A Karl Fischer (KF) measurement of the solution with the precipitate is determined to be about <6 wt %. In an additional embodiment, 1-3 vol, particularly 2 vol, of C₄H₁₀—O₅H₁₂ ether (1 wt (g) of dibenzyl ((ethane-1,2-diylbis(benzylazane-diyl))bis(ethane-2,1-diyl))dicarbamate dihydrochloride:1 volume (mL) C₄H₁₀-C₅H₁₂ ether) is added to the solution with the precipitate and stirred for about 1-3 hr, particularly 2 hr, to effect additional precipitation. The precipitate is isolated, e.g., by filtering or centrifuging, particularly filtering. In one embodiment, the C₄H₁₀—O₅H₁₂ ether is tert-butyl methyl ether (TBME). In another embodiment, isolated precipitate, trientine dihydrochloride, is rinsed at least once, particularly twice, with 1 vol of C₄H₁₀—O₅H₁₂ ether. In one embodiment, the C₄H₁₀—O₅H₁₂ ether is TBME. The trientine dihydrochloride is dried at about 20-30° C., particularly at about 25° C. In another embodiment, the drying is optionally also conducted under vacuum. In a further embodiment, the drying is carried out for about 1 hour to 5 days, particularly for about 1-2 hr.

The invention also includes the embodiment wherein the trientine dihydrochloride (1 eq.) is recrystallized in a C₁₋₅ alcohol and water mixture. In one embodiment, the recrystallization occurs in about 9-12 vol, particularly 10.5 vol, of C₁₋₅ alcohol and water mixture per 1 wt (g) of trientine dihydrochloride at about 40-60° C.; particularly between about 50-60° C., more particularly between about 45-50° C. In another embodiment, the C₁₋₅ alcohol and water mixture is at a wt % of C₁₋₅ alcohol in water of about 5-20 wt %, particularly about 5-10% C₁₋₅ alcohol to water, more particularly about 7%. In one embodiment, the recrystallization mixture is cooled to about 10-25° C., particularly to about 15-20° C., more particularly to about 10-15° C. for about 30 min-2 hr, particularly about 1 hr. Trientine dihydrochloride may begin to precipitate at about 40° C. In another embodiment, an antisolvent may be added to the recrystallization mixture. In another embodiment, the antisolvent is a C₄H₁₀—O₅H₁₂ ether, particularly TBME. In yet another embodiment, the C₄H₁₀-C₅H₁₂ ether is added in about 2-5 vol, particularly about 3.3 vol, per wt (g) of trientine dihydrochloride. In another embodiment, the reaction mixture with the added antisolvent is held for an additional 1-4 hr, particularly about 2 hr. Precipitate is then isolated, for example, by filtration, and optionally washed once or twice with antisolvent (about 2-6 vol, particularly about 4 vol, per wt (g) of trientine dihydrochloride), particularly TBME, and dried at 20-30° C., particularly at about 25° C. In another embodiment, the drying is optionally also conducted under vacuum. In a further embodiment, the drying is carried out for about 1 hour to about 5 days, particularly for about 1-2 hr. Recrystallized trientine dihydrochloride is a white solid.

Recrystallized trientine dihydrochloride is produced in high purity. In one embodiment, it is ≥95%, particularly ≥97.5%, and more particularly ≥99% pure. In another embodiment, the trientine dihydrochloride may comprise not more than (NMT) about 0.5% of any single impurity. In another embodiment, the trientine dihydrochloride may comprise NMT about 0.15 wt % of any single known impurity and NMT about 0.10 wt % of any unknown impurity.

Unless context dictates otherwise, the descriptions and definitions of the features set out above are not limited to any particular aspect or embodiment of the invention and apply equally to all aspects and embodiments which are described.

### EXAMPLES

### Example 1—Preparation of Dibenzyl ((Ethane-1,2-Diylbis(Benzylazanediyl))Bis(Ethane-2,1-Diyl))Dicarbamate Dihydrochloride

To a suspension of CbzOSu (2.5 eq.) in toluene (3 vol) at about 15-20° C. is added aqueous 2-chloroethylamine HCl (70 wt %, 2.75 eq.), followed by the dropwise addition of an 11.5 wt % solution of sodium carbonate (2.9 eq.) while maintaining the temperature below about 25° C. Upon completion of the addition, the batch is stirred at about 15-25° C. for an additional 2 hr, at which in process control (IPC) testing by UPLC or HPLC of the organic aromatic layer is used to confirm the preparation of benzyl (2-chloroethyl)carbamate (CBzHNethylchloride). The organic aromatic layer is separated and set aside from the aqueous layer.

N,N′-dibenzylethylenediamine diacetate (1 eq.), the organic aromatic layer containing benzyl (2-chloroethyl)carbamate (CBzHNethylchloride) from the previous reaction, water (3 vol) are added to a reaction vessel followed by the addition of 15.3 wt % aq. sodium carbonate (2.3 eq.), and CO₂ evolution is observed. This mixture is heated to about 85-90° C. to distill off toluene until the reaction mixture temperature reaches about 100° C. Once the toluene is removed, the mixture is refluxed and stirred for about 20 h at this temperature. IPC using UPLC or HPLC is also taken of the mixture to confirm the preparation of dibenzyl ((ethane-1,2-diylbis-(benzylazanediyl))bis(ethane-2,1-diyl))dicarbamate.

The reaction is cooled to 30° C., then toluene (7 vol) is added and the reaction is cooled to 20° C. The aqueous layer is removed and disposed of. To the organic layer is added ethanol (7 vol) and the mixture is cooled to 0-5° C. Aqueous HCl 37 wt % (2.2 equiv) is added maintaining<10° C. and a white precipitate forms. The mixture is stirred an additional 2 h at 0-5° C. and then is filtered. To the vessel is added 48 wt % EtOH in PhMe (2 vol), which is stirred for no less than 5 minutes and then charged onto the cake as a rinse. Two additional rinses (2 vol each) of solvent 48 wt % EtOH in PhMe are charged and filtered, at which point the solid is dried under vacuum at 55° C. to yield dibenzyl ((ethane-1,2-diylbis(benzylazanediyl))bis(ethane-2,1-diyl))dicarbamate dihydrochloride as a white solid. Note: All vol amounts are based on input of N,N′-dibenzylethylenediamine diacetate, i.e., an eq amount of N,N′-dibenzylethylenediamine diacetate is equal to X g of N,N′-dibenzyl-ethylenediamine diacetate that is in turn equal to X ml (1 vol) of the desired solvent or solution.

### Example 2—Preparation of Trientine Dihydrochloride

To a suspension of dibenzyl ((ethane-1,2-diylbis(benzylazanediyl))bis(ethane-2,1-diyl))dicarbamate dihydrochloride (1 eq.), Pd/C 10% w/50% H₂O (0.4 wt % anhydrous basis, 0.25 mol % Pd) in 30 wt % water in EtOH (6.67 vol) is hydrogenated in a Hastelloy pressure vessel under 3 atm at 20-25° C. for 6 hr, at which point IPC using UPLC-CAD is taken to confirm reaction completion. The reaction mixture is filtered through a 0.2 μm capsule and rinsed with 30 wt % water in EtOH (2×1 vol) and a sample is obtained to analyse for trace palladium by FIO Pd using ICP-OES.

To the filtrate is added a metal scavenger, Quadrasil MP (6.67 wt %, relative to the theoretical yield of the product trientine dihydrochloride in grams), and the batch is stirred for 18 hr, at which point a second FIO ICP sample is obtained (ICP-OES). After, the batch is polish filtered (0.45 μm), concentrated to 3 vol, then it is diluted to 9 vol with EtOH, and then concentrated again. The process of concentration and dilution and concentration is repeated three more times and then after the last concentration, a precipitate is formed and isolated. The solution containing the precipitate is determined to have a KF of <6 wt %. TBME (2 vol) is added to the solution with the precipitate and the mixture is stirred for 2 hr. At this point, the mixture is filtered and isolated precipitate is rinsed with TBME (2×1 vol). The precipitate, trientine, is dried at 25° C. Note: all vol based on input of dibenzyl ((ethane-1,2-diylbis(benzylazanediyl))bis(ethane-2,1-diyl))dicarbamate dihydro-chloride, i.e., an eq amount of dibenzyl ((ethane-1,2-diylbis(benzylazanediyl))bis(ethane-2,1-diyl))dicarbamate dihydrochloride is equal to x g of dibenzyl ((ethane-1,2-diylbis(benzylazanediyl))bis(ethane-2,1-diyl))dicarbamate dihydrochloride that is in turn equal to x ml (1 vol) of the desired solvent/solution.

### Example 3—Trientine Dihydrochloride Recrystallization

Trientine dihydrochloride from Example 2 is suspended (1 eq.) in 7 wt % water in EtOH (10.5 vol) at 15-20° C., and then is heated until full dissolution, about 45-50° C. The solution is then cooled to 15-20° C., with trientine dihydrochloride beginning to precipitate at about 40° C. The batch is held at 15-20° C. for 1 hr, at which point TBME (3.3 vol) is added and the solution is held an additional 2 hr. At this point, the solution is filtered to isolate precipitated trientine dihydrochloride, the recrystallization vessel is rinsed using TBME (4 vol), and the rinse is used in two separate portions to wash the isolated trientine dihydrochloride. The trientine dihydrochloride is dried at 25° C. and is a white solid. Note: all vol based on input trientine dihydrochloride, i.e., an eq amount of trientine dihydrochloride is equal to X g of trientine dihydrochloride that is in turn equal to X ml (1 vol) of the antisolvent.

1. A process for preparing dibenzyl ((ethane-1,2-diylbis(benzylazanediyl))bis(ethane-2,1-diyl))dicarbamate comprising the step of: reacting benzyl (2-chloroethyl)carbamate with N,N′-dibenzylethylenediamine.

2. The process of claim 1, wherein the benzyl (2-chloroethyl)carbamate is dissolved in an aromatic solvent.

3. A process of claim 2, wherein water is present as an additional solvent.

4. The process of claim of 1, wherein the N,N′-dibenzylethylenediamine is generated in situ.

5. The process of claim 4, wherein the N,N′-dibenzylethylenediamine is generated in situ using an acid addition salt of N,N′-dibenzylethylenediamine and an inorganic base selected from potassium hydroxide, sodium hydroxide, calcium hydroxide, magnesium hydroxide, potassium carbonate, sodium carbonate, calcium carbonate and magnesium carbonate.

6. The process of claim 5, wherein the acid addition salt of N,N′-dibenzylethylenediamine is N,N′-dibenzylethylenediamine diacetate.

7. The process of claim 6, wherein the inorganic base inorganic base is in the form of an aqueous solution.

8. The process of claim 7, wherein the inorganic base is sodium carbonate.

9. The process of claim 7, wherein the aqueous solution of the inorganic base is 10-20 wt % of the base.

10. The process of claim 7, wherein the molar equivalents ratio of the acid addition salt of N,N′-di-benzylethylenediamine:benzyl (2-chloroethyl)carbamate:inorganic base is about 1:2-2.6:2.2-2.6.

11. The process of claim 7, wherein the aromatic solvent to total water volume ratio is about 1:1-2.

12. The process of claim 2, wherein the reaction is heated to about 80-100° C.

13. The process of claim 12, wherein the the heating is maintained to distill off the aromatic solvent.

14. The process of claim 13, wherein the aromatic solvent is considered distilled off when reaction mixture temperature reaches about 100° C.

15. The process of claim 14, wherein following the distilling off of the aromatic solvent, the residual aqueous solution is stirred for about 15-30 hr at about 100° C.

16. The process of claim 15, wherein further comprising the step of: cooling the residual aqueous solution to about 20-40° C.

17. The process of claim 16, wherein additional aromatic solvent is added to the residual aqueous solution.

18. The process of claim 17, wherein the aromatic and aqueous solvent phases are separated and the aqueous solvent phase is discarded.

19. The process of claim 7, further comprising the step of: acidifying the product with HCl to form the dihydrochloride salt.

20.-62. (canceled)
